(19) United States
(12) Patent Application Publication
(10) Pub. No.: US 2024/0016655 A1
(43) Pub. Date: Jan. 18, 2024

(54) DEVICE AND METHOD FOR PROTECTING A FETUS FROM INJURY DURING UMBILICAL CORD PROLAPSE

(71) Applicant: COOK MEDICAL TECHNOLOGIES LLC, Bloomington, IN (US)

(72) Inventors: Qing Qing Wang, Bloomington, IN (US); Parker Bassett, Bloomington, IN (US); Jayton Buxkemper, Bloomington, IN (US); Megan O'Keefe, Louisville, KY (US)

(73) Assignee: COOK MEDICAL TECHNOLOGIES LLC, Bloomington, IN (US)

(21) Appl. No.: 18/350,052

(22) Filed: Jul. 11, 2023

Related U.S. Application Data
(60) Provisional application No. 63/388,341, filed on Jul. 12, 2022.

(51) Int. Cl. A61F 6/12; A61F 6/16; A61F 6/18

(52) U.S. Cl. CPC A61F 6/12; A61F 6/16; A61F 6/18

(57) ABSTRACT

A method of protecting a fetus from injury during umbilical cord prolapse includes delivering a device which includes a hollow sheath and a compliant portion partially or completely surrounding the hollow sheath into a vaginal canal of a patient. The device is maneuvered within the vaginal canal and/or cervical opening such that a prolapsed umbilical cord enters the hollow sheath. The device is then positioned within a uterus of the patient, such that a first face of the compliant portion contacts a fetus and a second face of the compliant portion contacts a wall of the uterus.

### RELATED APPLICATION

The present patent document claims the benefit of priority under 35 U.S.C. 119(e) to U.S. Provisional Patent Application No. 63/388,341, which was filed on Jul. 12, 2022, and is hereby incorporated by reference in its entirety.

### TECHNICAL FIELD

The present disclosure is related generally to a medical device for use during childbirth and more particularly to a device and method for removing pressure from the umbilical cord during umbilical cord prolapse.

### BACKGROUND

Umbilical cord prolapse is an acute obstetric emergency that may occur prior to or during childbirth. The complication occurs when the umbilical cord drops through the open cervix, into the vaginal canal, before the fetus. The cord may become compressed, causing diminished blood flow and oxygen supply to the fetus. This may lead to brain damage, short episodes of fetal hypoxia, and even death. Some common risk factors for umbilical cord prolapse include preterm labor, premature rupture of membranes, multiple gestation pregnancies, polyhydramnios, and malpresentation of the fetus. After identifying prolapse of the cord, treatment options typically involve manually relieving the pressure on the umbilical cord and performing an emergency C-section. The current practice is for the medical practitioner to lift the presenting part of the fetus away from the umbilical cord with his or her fingers until the patient is ready for the C-section. A better solution is needed to minimize the risks associated with this complication.

### BRIEF SUMMARY

A device and method for protecting a fetus from injury during umbilical cord prolapse are described.

The device comprises a hollow sheath configured to resist compressive forces and to contain part or all of a prolapsed umbilical cord, and a compliant portion partially or completely surrounding the hollow sheath. The compliant portion is configured to contact a fetus and a uterine wall without causing injury.

The method comprises delivering a device which includes a hollow sheath and a compliant portion partially or completely surrounding the hollow sheath into a vaginal canal of a patient. The device is maneuvered within the vaginal canal and/or cervical opening such that a prolapsed umbilical cord enters the hollow sheath. The device is then positioned within a uterus of the patient, such that a first face of the compliant portion contacts a fetus and a second face of the compliant portion contacts a wall of the uterus.

### BRIEF DESCRIPTION OF THE DRAWINGS

FIG. 1 shows the device according to an embodiment where the compliant portion comprises inflatable balloons.

FIG. 2 shows the device according to an embodiment where the inflatable balloons are separately inflatable.

FIG. 3 shows the device according to an embodiment where the hollow sheath has a two-piece construction.

FIG. 4 shows the device according to an embodiment where the compliant portion comprises fluid-filled bubble regions.

FIG. 5 shows the device according to an embodiment where the compliant portion comprises a polymeric coating.

FIG. 6 shows the device according to an embodiment where the compliant portion comprises a foam.

FIG. 7 illustrates placement of the device in a patient's body.

### DETAILED DESCRIPTION

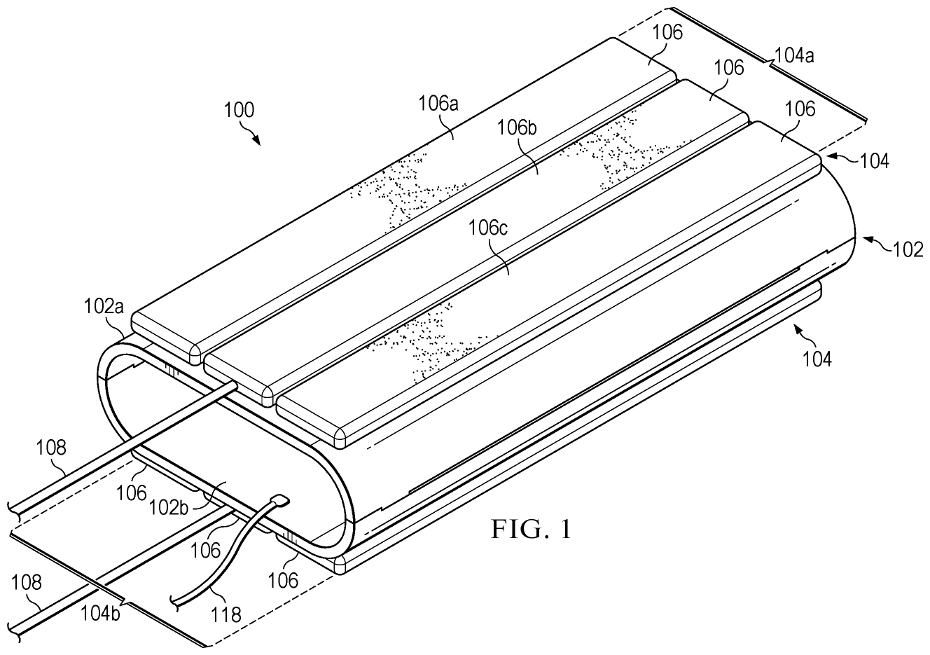

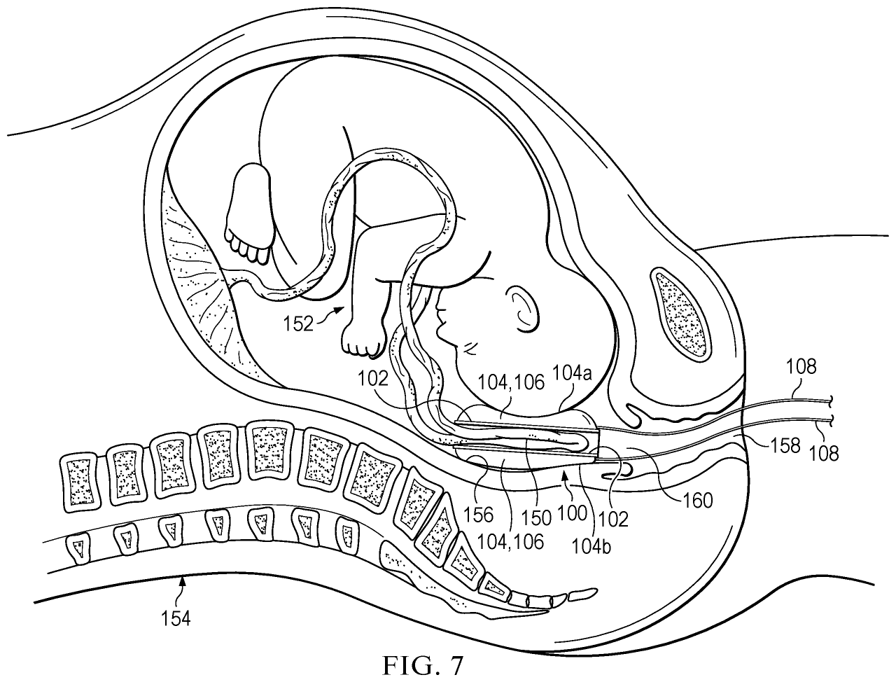

A device 100 for protecting a fetus from injury during umbilical cord prolapse is shown according to various embodiments in FIGS. 1-6 and is described below. FIG. 7 shows a cross-sectional view of the device 100 positioned in a patient's uterus. The inventive device 100 may enable medical practitioners (e.g., physicians, nurses) to protect a prolapsed umbilical cord while freeing up the medical practitioner's hand to address other issues during childbirth. Advantageously, the device 100 is engineered to prevent compression of the umbilical cord—such that blood, nutrients and oxygen may be continuously supplied to the fetus—and to contact the fetus and uterine wall without causing harm. Use of the device 100 may result in a better prognosis for both the mother and the fetus.

A method of using the device 100, including inserting the device 100 into the patient's body, maneuvering the device 100 to capture the prolapsed umbilical cord, and positioning the device 100 within the uterus, is described below. First, various embodiments of the device 100 as shown in FIGS. 1-6 are discussed.

The device 100 includes a hollow sheath 102 and a compliant portion 104 partially or completely surrounding the hollow sheath 102. The hollow sheath 102 is configured to resist compressive forces and to contain part or all of a prolapsed umbilical cord 150, as illustrated in FIG. 7. The compliant portion 104 that overlies the hollow sheath 102 is configured to contact the fetus 152 and an opposing part of the uterine wall 156 without inflicting injury. The compliant portion 104 serves as a soft interface between the stiffer hollow sheath 102 and the anatomy of the fetus 152 and patient 154. Accordingly, the compliant portion 104 may be engineered to absorb compressive forces exerted by the fetus 152 and the uterine wall 156, which may be undergoing contractions, while the device 100 is in use. As discussed below, the compliant portion 104 may comprise inflatable balloons, fluid-filled bubble regions, a coating, a foam, etc. The device 100 may be effectively anchored in place by contact with the fetus 152 and the uterine wall 156; however, to ensure that the device 100 does not move once placed, the device 100 may further include a tether 118 (shown in FIG. 1) attached to the hollow sheath 102 that has a length sufficient to extend through the vaginal canal and be secured outside the patient's body, e.g., a length from about 20 cm to about 40 cm. Due to the use of the device 100 inside a patient's body, the hollow sheath 102, the compliant portion 104, and any other component(s) of the device 100 are advantageously formed from biocompatible materials.

Referring to FIG. 1, it may be beneficial for the hollow sheath 102 to have a flattened tubular shape with first and second sides 102a,102b. The compliant portion 104 may cover the first and second sides 102a,102b to provide a first face 104a to contact the fetus and a second face 104b to contact the opposing cervical or uterine wall. The compliant portion 104 may have a single-piece construction or a multi-piece construction (e.g., a two-piece construction, as illustrated). The flattened tubular shape of the hollow sheath 102 has an oblong cross-section that can accommodate the outgoing and returning sections of an umbilical cord that has dropped into or through the cervical opening. It is also contemplated that the hollow sheath 102 may have a (regular) tubular shape with a circular cross-section. A typical umbilical cord 150 has a diameter in a range from about 1 cm to 2 cm, and thus the hollow sheath 102 preferably has a diameter or inner width in a range from about 2 cm to about 4 cm. If the hollow sheath 102 has a flattened tubular shape, the long dimension of the oblong cross-section (or the inner length, which is normal to the inner width), may range in size from about 3 cm to about 6 cm. The hollow sheath 102 may have a total length in a range from about 7.5 cm to about 13 cm, where the total length is measured along a longitudinal axis of the sheath 102.

To resist compressive forces while being delivered and positioned within the body, the hollow sheath 102 may have a stiffness or flexural modulus of at least about 1 GPa, at least about 1.5 GPa, or at least about 2 GPa. The hollow sheath 102 may be formed from one or more biocompatible polymers, such as polycarbonate, polyethylene, polypropylene, and/or acetal, also known as polyoxymethylene. Polycarbonate has a flexural modulus of about 2.3 GPa; polyethylene (high density polyethylene or HDPE) has a flexural modulus of about 1.2 GPa; polypropylene has a flexural modulus of about 1.5 GPa, and acetal has a flexural modulus of about 2.9 GPa. While the mechanical properties of the hollow sheath 102 may be sufficient to protect the prolapsed umbilical cord, the sheath 102 is not highly rigid and may exhibit some flexure in use. The hollow sheath 102 may be substantially straight along the longitudinal direction, as illustrated in FIG. 1, or curved, e.g., to better accommodate the anatomy of the patient 154 or fetus 152.

As indicated above, the compliant portion 104 is engineered to absorb compressive forces. Advantageously, when forces (e.g., uterine contractions) are applied, the compliant portion 104 conforms to the adjacent anatomy to disperse the forces over a larger area. Preferably, the compliant portion 104 is fabricated from a biocompatible polymer. Shore durometer, per the ASTM D2240 standard, provides a measure of hardness (or softness) of a polymer, and for this application, the biocompatible polymer may have a Shore A scale hardness (or “Shore durometer”) in a range from about 10 to about 50, or from about 10 to about 30. The compliant portion 104 may completely cover the exterior surface of the hollow sheath 102 or may be applied to just a portion of the exterior surface, e.g., to the first and second sides 102a,102b of the hollow sheath 102, as shown in FIGS. 1-3, for example.

The compliant portion 104 may comprise one or more inflatable balloons 106, such as at least two balloons or at least four balloons, and up to six balloons, up to eight balloons, or up to ten balloons. The balloons may be positioned on the first and second sides 102a,102b of the hollow sheath 102. In the example of FIG. 1, the compliant portion 104 includes three inflatable balloons 106 positioned on the first side 102a and three inflatable balloons positioned on the second side 102b of the sheath 102. The device 100 may further include one or more inflation lumens 108 (two in this example) in fluid communication with the inflatable balloons 106, as illustrated. It is noted that components described as being in fluid communication with each other may be understood to be connected directly or indirectly such that fluid can flow in one or both directions between and/or through the components. The one or more inflation lumens 108 may be configured for connection to an external source of an inflation fluid, e.g., to a syringe and/or saline bag outside the patient's body. In this example, each inflation lumen 108 inflates multiple balloons 106, specifically, the three balloons 106a,106b,106c on each side 102a,102b of the hollow sheath 102. The inflation lumen 108 splits into channels (not shown) configured to deliver the inflation fluid to each of the balloons 106a,106b,106c. Each inflation lumen 108 has a length sufficient to extend from the internal position of the device 100 to the external source of inflation fluid. After the device 100 is delivered into the patient's body, the inflatable balloons 106 in fluid communication with the inflation lumen 108 may be (simultaneously) inflated as inflation fluid is pumped from the external source through the inflation lumen 108.

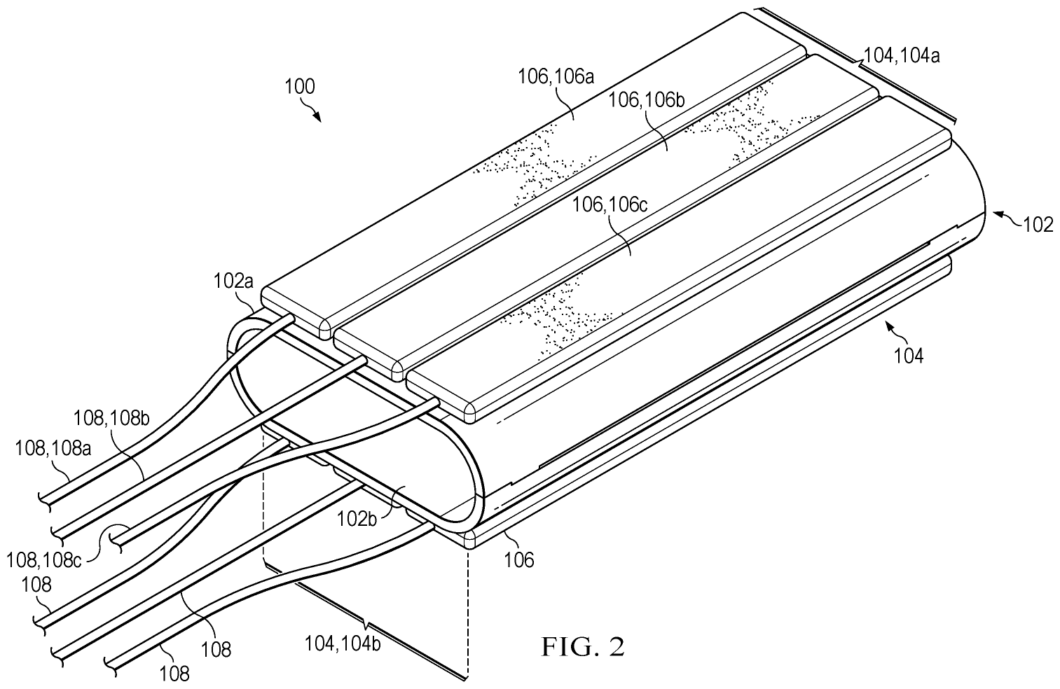

Referring now to FIG. 2, an alternative embodiment is illustrated where each inflatable balloon 106 is individually connected to an inflation lumen 108. In such an example, after delivery of the device 100 into the patient's body, the inflatable balloons 106 may be independently inflated as inflation fluid from the external source is pumped through the respective lumen 108. For example, the central balloon 106b may be selectively inflated by pumping inflation fluid through the middle inflation lumen 108b. In the examples of FIGS. 1 and 2, the inflation lumens may have a length sufficient to extend from the internal position of the device 100, through the vaginal canal and to the external source of inflation fluid, e.g., a length in a range from about 20 cm to about 40 cm. Each inflation lumen 108 may be formed of biomedical grade silicone or another polymer. An advantage of using a compliant portion 104 comprising one or more inflatable balloons 106 as shown in FIGS. 1 and 2 is that the size of the device 100 may be reduced for delivery into the patient's body (since the balloons 106 are initially deflated). In addition, a compliant portion 104 including multiple balloons 106 with independently controlled inflation lumens 108 can be selectively inflated, e.g., according to the position of the fetus.

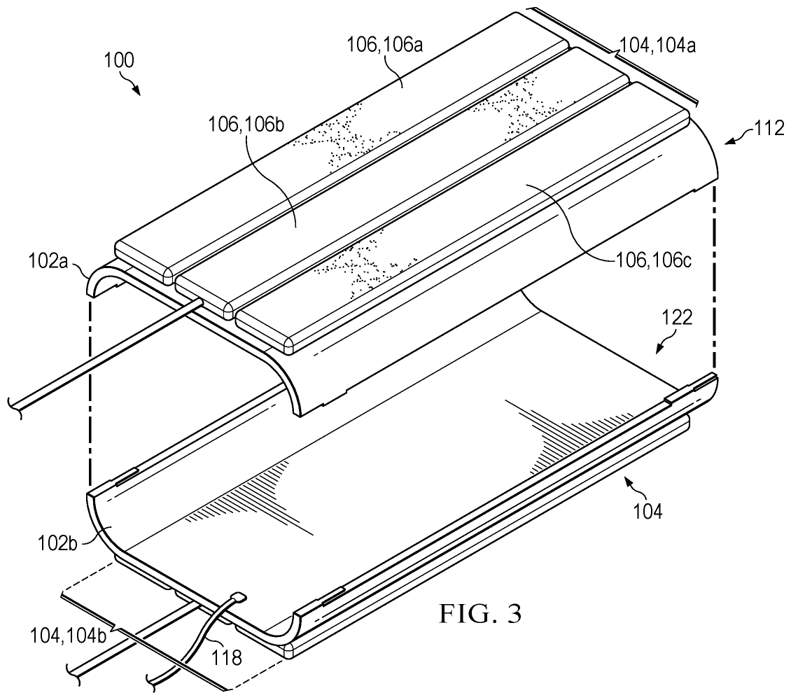

The hollow sheath 102 may have a single-piece or a multi-piece construction, as shown for example in FIG. 3. In the latter case, the hollow sheath 102 may be engineered to open and close, e.g., by splitting into two longitudinal sections 112,122 that may be reconnected in use. The device 100 may be joined together by any of various fastening approaches known in the art, such as a friction fit, snap fit, press fit, or magnetic attachment.

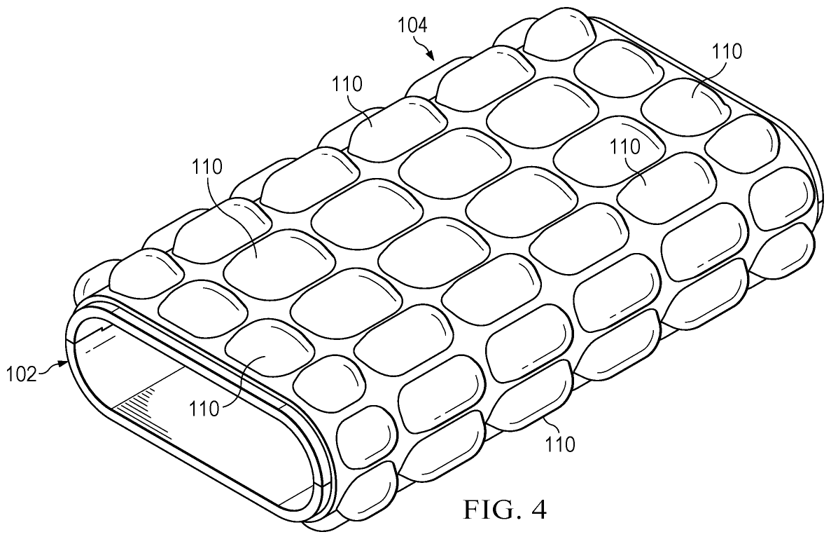

Referring now to FIG. 4, which shows another embodiment of the device 100, the compliant portion 104 may include one or more fluid-filled bubble regions 110 that are “pre-inflated” and thus do not require inflation after delivery into the patient's body. In this example, no inflation lumens are required, and each of the bubble regions 110 is sealed to prevent leakage of the fluid. The compliant portion 104 may include a plurality of the fluid-filled bubble regions 110, which may partially or fully surround the exterior surface of the device 100. For example, the compliant portion 104 may include at least 20, at least 50, or at least 100 of the fluid-filled bubble regions.

The one or more inflatable balloons 106 described above and/or the bubble regions 110 may be made from a biocompatible polymer, such as a thermoplastic elastomer. Suitable polymers may include, for example, polysiloxane (silicone), polyethylene teraphthalate (PET), polyethylene, nylon, and/or a block copolymer of polyether glycol and polybutylene terephthalate (PBT). The inflation fluid employed to inflate the inflatable balloons 106 and/or to fill the bubble regions 110 typically comprises a saline solution or air. In some examples the fluid may comprise a radiopaque solution that includes a contrast medium dispersed in the saline solution.

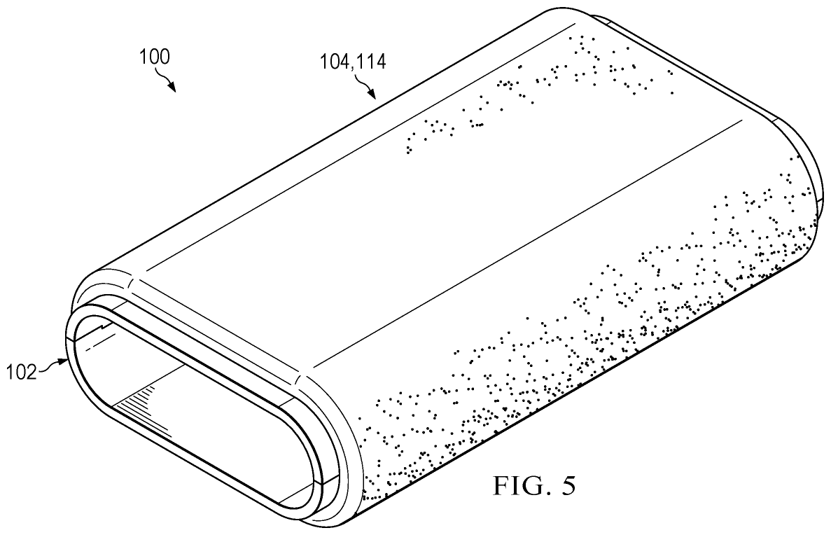

Referring now to FIG. 5, in another embodiment of the device 100, the compliant portion 104 may comprise a polymeric coating 114 formed from one or more elastomers, such as medical grade silicone. Preferably, the polymeric coating 114 has a substantial thickness in a range from about 1 mm to about 10 mm for absorption of compressive forces. The polymeric coating 114 may comprise a single layer or multiple layers. As indicated above, the polymeric coating 114 may completely surround the hollow sheath 102 or may only partially cover the hollow sheath 102.

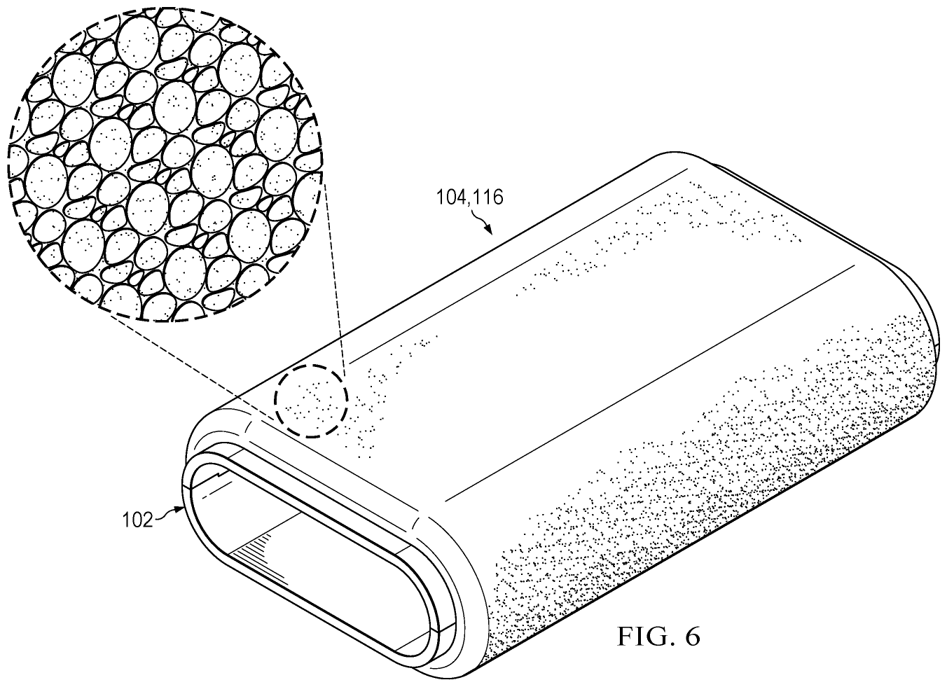

Referring now to FIG. 6, in another embodiment of the device 100, the compliant portion 104 may comprise a foam 116, such as a closed-cell foam. Suitable examples may include a polyethylene foam, e.g., a cross-linked polyethylene foam. The foam 116 may completely surround the hollow sheath 102, as illustrated, or may only partially cover the hollow sheath 102.

Now that various embodiments of the device 100 have been described, a method of using the device 100 to protect a fetus from injury during umbilical cord prolapse is explained in reference to FIG. 7. The device 100, which includes a hollow sheath 102 and a compliant portion 104 partially or completely surrounding the hollow sheath 102, as described above, is inserted into a patient's vaginal canal 158. The insertion may be carried out manually by a medical practitioner. It is understood that the device 100 employed in the method may have any of the components, features, materials, and/or properties described above or elsewhere in this disclosure.

Once inserted, the device 100 is maneuvered within the vaginal canal 158 and/or cervical opening 160 such that a prolapsed umbilical cord 150 enters the hollow sheath 102. The device 100 is then positioned within the uterus such that a first face 104a of the compliant portion 104 contacts the fetus 152 and a second face 104b of the compliant portion 104 contacts the uterine wall 156, as shown in FIG. 7. The device 100 may be effectively anchored in place between the fetus 152 and the uterine wall 156 as the head (or other body part) of the fetus 152 compresses the first face 104a. If desired, a tether 118 attached to the hollow sheath 102, as described above in reference to FIG. 1, may be secured outside the patient's body to prevent or minimize unwanted distal or proximal motion of the device 100, once positioned. Also or alternatively, the inflation lumen(s) 108 may be employed as a tether. The hollow sheath 102 is configured as described above to resist compressive forces such that the prolapsed umbilical cord 150, which is partially or completely contained within the sheath 102, is not compressed and continues to supply blood, nutrients and oxygen to the fetus 152. The compliant portion 104 is engineered as described above to avoid injuring the fetus 152 and the uterine wall 156 while the device 100 is in use.

The maneuvering of the device 100 to enable entry of the prolapsed cord 150 into the hollow sheath 102 may entail sliding the hollow sheath 102 in a distal direction through the vaginal canal 158 and/or the cervical opening 160 while targeting the prolapsed cord 150. This approach may be particularly suitable when the device 100 has a single-piece construction. In some examples, the device 100 may have a multi-piece construction and/or be configured to open and close, as described above, and thus the maneuvering may entail: (a) positioning the device 100 adjacent to the prolapsed umbilical cord 150, (b) opening the device 100 to split the hollow sheath 102 into two longitudinal sections 112,122, as shown in FIG. 3, (c) surrounding the prolapsed umbilical cord 150 with the two longitudinal sections 112,122, and (d) closing the device 100 about the prolapsed umbilical cord 150 (e.g., bringing together and/or fastening the two longitudinal sections 112,122). This action may be carried out manually by the medical practitioner.

In an example where the compliant portion 104 comprises one or more inflatable balloons 106 and the device 100 further comprises one or more inflation lumens 108 in fluid communication with the one or more inflatable balloons 106, as described above, after delivering the device 100 into the patient's vaginal canal 158, an inflation fluid may be pumped through the one or more inflation lumens 108 to inflate the one or more balloons 106. The inflatable balloons 106 may be inflated before or after the device 100 is maneuvered to partly or completely contain the prolapsed cord 150. Preferably, the inflatable balloons 106 are inflated before the device 100 is positioned in contact with the fetus 152 and the uterine wall 156. In an example where the compliant portion 104 comprises a plurality of inflatable balloons 106 and the device further comprises one or more inflation lumens 108 in fluid communication with the plurality of inflatable balloons 106, as described above, prior to positioning the device 100 within the uterus, an inflation fluid may be pumped through the one or more inflation lumens 108 to simultaneously inflate the plurality of inflatable balloons. As indicated above, an advantage of using a compliant portion 104 comprising one or more inflatable balloons 106 is that the size of the device 100 may be reduced for delivery into the patient's body (since the balloons 106 are initially deflated). In addition, if the inflatable balloons 106 have inflation lumens 108 that are independently controlled, then the balloons 106 can be selectively inflated, e.g., according to the position of the fetus 152, while in use. In other words, in an example where the compliant portion 104 comprises a plurality of inflatable balloons 106 and the device 100 further comprises a plurality of inflation lumens 108 in fluid communication with the plurality of inflatable balloons 106 (e.g., as shown in FIG. 2), prior to positioning the device within the uterus, an inflation fluid may be pumped through a predetermined inflation lumen from the plurality of inflation lumens 108 to selectively inflate a predetermined inflatable balloon from the plurality of inflatable balloons 106.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein.

Furthermore, the advantages described above are not necessarily the only advantages of the invention, and it is not necessarily expected that all of the described advantages will be achieved with every embodiment of the invention.

1. A device for protecting a fetus from injury during umbilical cord prolapse, the device comprising:
a hollow sheath configured to resist compressive forces and to contain part or all of a prolapsed umbilical cord; and
a compliant portion partially or completely surrounding the hollow sheath, the compliant portion being configured to contact a fetus and a uterine wall without causing injury.

2. The device of claim 1, wherein the compliant portion comprises a biocompatible polymer having a Shore A scale hardness in a range from about to about 50, and/or
wherein the hollow sheath has a flexural modulus of at least about 1 GPa.

3. The device of claim 1, wherein the hollow sheath comprises a biocompatible polymer selected from the group consisting of: polycarbonate, polyethylene, polypropylene, and acetal.

4. The device of claim 1, wherein the hollow sheath has a flattened tubular shape with an oblong cross-section.

5. The device of claim 1, wherein the hollow sheath has a tubular shape with a circular cross-section.

6. The device of claim 1, wherein the hollow sheath has an inner diameter or inner width in a range from about 2 cm to about 4 cm, and/or
wherein the hollow sheath has a length in a range from about 7.5 cm to about 13 cm.

7. The device of claim 1, wherein the compliant portion comprises one or more inflatable balloons, and wherein the device further comprises one or more inflation lumens in fluid communication with the one or more inflatable balloons and configured for connection to an external source of inflation fluid.

8. The device of claim 1, wherein the compliant portion comprises one or more fluid-filled bubble regions, each of the fluid-filled bubble regions being sealed to prevent leakage of the fluid.

9. The device of claim 1, wherein the compliant portion comprises a foam.

10. The device of claim 1, wherein the compliant portion comprises a polymeric coating including an elastomer.

11. The device of claim 1, wherein the hollow sheath has a single-piece construction.

12. The device of claim 1, wherein the hollow sheath has a multi-piece construction.

13. The device of claim 1, further comprising a tether attached to the hollow sheath, the tether having a length sufficient to be secured outside a patient's body while in use.

14. A method of protecting a fetus from injury during umbilical cord prolapse, the method comprising:
delivering a device into a vaginal canal of a patient, the device comprising a hollow sheath and a compliant portion partially or completely surrounding the hollow sheath;
maneuvering the device within the vaginal canal and/or cervical opening such that a prolapsed umbilical cord enters the hollow sheath; and
positioning the device within a uterus of the patient, whereby a first face of the compliant portion contacts a fetus and a second face of the compliant portion contacts a wall of the uterus.

15. The method of claim 14, wherein the maneuvering comprises sliding the hollow sheath in a distal direction through the vaginal canal and/or the cervical opening.

16. The method of claim 14, wherein the device is configured to open and close, and
wherein the maneuvering comprises:
positioning the device adjacent to the prolapsed umbilical cord;
opening the device to split the hollow sheath into two longitudinal sections;
surrounding the prolapsed umbilical cord with the two longitudinal sections; and
closing the device about the prolapsed umbilical cord.

17. The method of claim 14, wherein the compliant portion comprises one or more inflatable balloons, one or more fluid-filled bubble regions, a polymeric coating, and/or a foam.

18. The method of claim 14, wherein the compliant portion comprises one or more inflatable balloons, and wherein the device further comprises one or more inflation lumens in fluid communication with the one or more inflatable balloons and extending through the patient's vaginal canal to an external source of inflation fluid,
further comprising, prior to positioning the device within the uterus, pumping an inflation fluid through the one or more inflation lumens, thereby inflating the one or more inflatable balloons.

19. The method of claim 14, wherein the compliant portion comprises a plurality of inflatable balloons, and wherein the device further comprises one or more inflation lumens in fluid communication with the plurality of inflatable balloons and extending through the patient's vaginal canal to an external source of inflation fluid,
further comprising, prior to positioning the device within the uterus, pumping an inflation fluid through the one or more inflation lumens, thereby simultaneously inflating the plurality of inflatable balloons.

20. The method of claim 14, wherein the compliant portion comprises a plurality of inflatable balloons, and wherein the device further comprises a plurality of inflation lumens in fluid communication with the plurality of inflatable balloons and extending through the patient's vaginal canal to an external source of inflation fluid,
further comprising, prior to positioning the device within the uterus, pumping an inflation fluid through a predetermined inflation lumen from the plurality of inflation lumens, thereby selectively inflating a predetermined inflatable balloon from the plurality of inflatable balloons.
